(19) United States
(12) Patent Application Publication
(10) Pub. No.: US 2021/0046162 A1
(43) Pub. Date: Feb. 18, 2021

(54) Teriparatide Use for Bone Stress Fractures/Reactions

(71) Applicant: University of South Carolina, Columbia, SC (US)

(72) Inventors: James Benjamin Jackson, III, Irmo, SC (US); Kathryn M. Diemer, St. Louis, MO (US)

(73) Assignee: University of South Carolina, Columbia, SC (US)

(21) Appl. No.: 16/993,609

(22) Filed: Aug. 14, 2020

Related U.S. Application Data
(60) Provisional application No. 62/888,021, filed on Aug. 16, 2019.

(51) Int. Cl. A61K 38/29; A61P 19/08

(52) U.S. Cl. CPC A61K 38/29; A61P 19/08

(57) ABSTRACT

Treatments and methods of treatment using intermittent exposure to human parathyroid hormone to stimulate new bone formation via osteoblasts to provide non-surgical/non-operative, anabolic remedies for beneficial effects on bone density, bone microarchitecture, and bone geometry in both cancellous and cortical bones to more rapidly heal and prevent further bone stress injuries.

### GOVERNMENT SUPPORT STATEMENT

This invention was made with government support under W81XWH-19-2-0051 awarded by the Army Medical Research and Materiel Command. The government may have certain rights in this disclosure.

### BACKGROUND OF THE INVENTION

### 1) Field of the Invention

The present invention relates to nonoperative treatments and methods of treatment for employing

### 2) Description of Related Art

The physical demands of basic military training and active theater deployment contribute to repetitive weight loads on the lower extremities. An alarming number of basic trainees and infantry soldiers exposed to these loads are developing painful, activity-limiting bone stress injuries (BSIs). If not adequately detected and managed, the BSI may result in prolonged loss of availability for duty, and a potential for medical discharge is created.

An example of the high burden of these BSIs was illustrated in a 2009 report listing their incidence during U.S. Army basic combat training as ranging between 0.9% and 5.2% for males and 3.4% to 21.0% for females making it the most common reason for lost duty days and training recycles. The incidence of BSIs among infantry soldiers who are training, or in theater, has been reported to be 6.9% per year. Overall, BSIs cost the Defense Department as much as $100 million dollars/year in medical costs and lost duty time.

Fort Jackson, located in Columbia, S.C., is the largest military training facility in the world (48,000 basic training and 12,000 additional advanced training Soldiers every year), and is located less than 15 miles from the primary location of the Palmetto Health University of South Carolina Orthopedic Center. Fort Jackson, as the U.S. Army's main production center for Basic Combat Training, trains 50% of the Army's Basic Combat Training load and 60% of the women entering the Army each year (Fort Jackson website). Basic training recruits have the highest incidence of BSIs among any large population. These factors combine to make Fort Jackson the highest per capita rate of BSIs in the world, with as many as 12 DTSFs diagnosed per week, according to on base medical personnel at Fort Jackson. This risk to soldiers and military readiness has been identified and has led to the observation that, “Stress fracture cases have been reported since the late 1800s, and today are one of the most common and potentially debilitating overuse injury seen in military recruits, particularly in women” (Biberdorf). The military focus on this critical issue combined with our study teams' access to the most vulnerable population in the world makes Columbia the ideal study location for this project, and provides a substantial level of relevance to the project outcomes as well.

Physiology of Bone Stress Injuries

Bone stress injuries, often called stress fractures, can affect almost any bone of the body, but the majority (approximately 95%) occur in the lower extremity weight bearing bones such as the tibia, tarsals and metatarsals. BSIs occur when the bone undergoes repeated load application, and an imbalance between bone resorption and formation takes place.

Activities such as running create ground reaction forces that are between three and eight times greater than walking on the lower extremity skeletal system and the repetitive mechanical loading causes increased hydrostatic pressure. Bone, being a dynamic tissue in accordance with Wolff s law, remodels in response to physiologic stress. When loading outstrips the pace of bone formation BSIs occur.

Normal bone has two components; cortical (compact) and cancellous (trabecular) bone. Cortical bone, which makes up about 80% of the skeleton, is located in the diaphysis of long bones as well as the “shell” of cuboid-like bones including vertebral bodies and carpal or tarsal bones. However, cancellous bone is found at the epiphysis and metaphysis of long bones and in cuboidlike bones. Because of these intrinsic characteristics of the bone, cortical fractures have been described classically as abnormal stresses to normal bone, and cancellous fractures as normal stresses on abnormal bone. However, the metabolic turnover rate is eight times slower in cortical bone compared to cancellous bone. For this reason, BSIs most often occur in the cortical bone. The exact pathophysiology of stress fractures is not clear, and there are several theoretical models to explain their causal mechanism. A commonly cited theoretical model is below in FIG. 1.

The skeleton receives repetitive bouts of mechanical loading during exercise. This loading transfers into bone strain, and when the strain is high enough it can lead to bone deformation. The strain is measured by the change in length per unit length of a bone. This value is unit-less, and is often clinically referred to as micro strain (με). The difference between normal bone strains (400-1500με)) and those required to fracture the bone (10,000με)) is large, but repetitive strains lower than that needed to fracture the bone in a single loading situation may cause damage, which is often termed micro damage.

Bone damage is a natural and useful biological phenomenon, and serves as a stimulus to activate bone formation and remodeling. This process occurs in basic multicellular units by osteoblasts (bone forming cells) and osteoclasts (bone resorbing cells). However, osteoclastic resorption reaches its peak at three weeks, sooner than osteoblastic bone formation.

Osteoblastic bone formation can take 6 weeks to three months to form normal lamellar layers. This gap between more rapid bone absorption and slower bone production formation is associated with partial or incomplete bone stress injury. In some circumstances an imbalance between damage generation and its removal occurs. The subsequent accumulated damage may be responsible to initiate the bone stress injury pathology continuum: stress reaction to stress fracture to complete bone fracture. The magnitude and rate at which strain is introduced and the absolute number of loading cycles are determinants of damage formation. The damage formation is threshold dependent; thus, damage increases with increasing strain. In relation to strain rate, strains that are introduced in shorter periods, such as the rigors of basic training, produce greater damage.

Most low-risk stress fractures (femoral shaft, 2nd-4th metatarsals) usually heal successfully without complications with conservative measures and restriction of activity. Initially, a period with activity modification and relative rest is usually required for approximately two to six weeks, during which the bone can heal and repair the stress fractures while the stress loading provides mechanical stimulation. Symptoms and pain-free thresholds should be used as a guide to the amount of activity modification, considering that all activity should be carried out in a pain-free level. In more advanced cases, the patient may suffer pain with walking and/or weight bearing activity. This is followed by a period of low impact activities, such as swimming or biking. High impact activities can be started once the patient can carry out low impact activities for a long time without pain. A gradual return to full activities is the standard of care.

A high risk stress fracture, such as a diaphyseal tibial stress fracture (DTSF), is at risk of progressing to delayed union, nonunion or displaced complete fracture. The limited soft tissue coverage, posterior entry of many of the nutrient arteries of the mid-shaft of the tibia contributes to it having a more limited blood supply than other bones of the lower extremity. The decreased blood supply can limit the nutrients to the osteoclasts and osteoblasts which stimulate bone healing. The most common location of a DTSF is the anterior cortex of the tibia. This segment of the bone has the least soft tissue coverage and is the furthest from the nutrient arties that most commonly enter from the posterior aspect of the tibia. A poorer physiologic environment for healing combined with a high load concentration make this location common for BSIs. This environment also increases the propensity for a prolonged healing or progression to complete fracture.

Patients who fail non-operative management because of persistent pain, inability to continue activities or who have a complete fracture require surgical intervention. To avoid these serious complications, these fractures should be treated with more aggressive measures. Accordingly, it is an object of the present disclosure to provide an effective non-operative, pharmacological treatment to accelerate healing, which until recently, fracture healing could not be readily accelerated.

### SUMMARY OF THE INVENTION

The above objectives are accomplished according to the present disclosure by providing a treatment for bone stress injuries. The treatment may include administering a therapeutically effective amount of an endogenous human parathyroid hormone to stimulate new bone formation via osteoblasts and avoids operative treatment and is anabolic. Further, the endogenous human parathyroid hormone may comprise teriparatide. Again, the endogenous human parathyroid hormone may be administered in a dosage ranging from 5 mcg to 50 mcg. Yet again, the endogenous human parathyroid hormone may be administered intermittently. Still, the endogenous human parathyroid hormone may be administered at least once every twenty-four hours. Further again, the endogenous human parathyroid hormone may be administered subcutaneously. Still yet, the endogenous human parathyroid hormone may have an amino acid sequence as shown in SEQ ID NO:1. Further yet, the treatment provides improvements in bone density, bone microarchitecture and bone geometry in both cancellous and cortical bones.

In a further embodiment, a method for treatment of bone stress injuries is provided. The method may include administering a therapeutically effective amount of teriparatide to increase bone production and bone mass via osteoblasts and does not include operative treatment and is anabolic. Further, the method may promote bone formation through osteoblast stimulation. Yet still, the endogenous human parathyroid hormone is administered in a dosage ranging from 5 mcg to 50 mcg. Again, teriparatide may be administered intermittently. Further still, teriparatide may be administered at least once every twenty-four hours. Again yet, teriparatide may be administered subcutaneously. Yet further, teriparatide may have an amino acid sequence as shown in SEQ ID NO:1. Again further, the treatment may provide improvements in bone density, bone microarchitecture and bone geometry in both cancellous and cortical bones.

In a still further embodiment, a prophylactic method for protecting against new and recurrent bone stress injuries is provided. The method may include administering a therapeutically effective amount of an endogenous human parathyroid hormone while avoiding operative treatment and being anabolic. Further, the endogenous human parathyroid hormone is administered in a dosage ranging from 5 mcg to 50 mcg. Further, teriparatide may be administered subcutaneously and intermittently. Yet still, teriparatide may be administered at least once every twenty-four hours.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows a proposed pathophysiology of stress fractures.

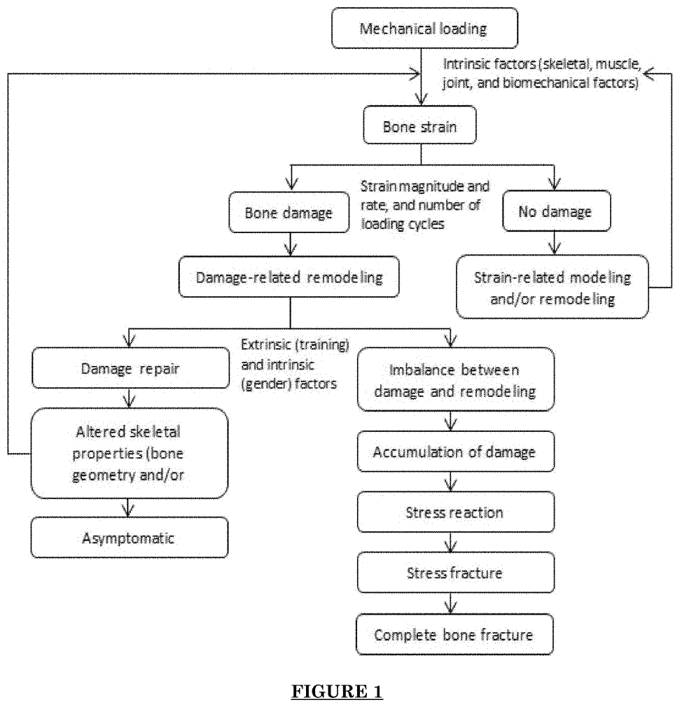

FIG. 2 shows a projected DTSF analysis study for the current disclosure.

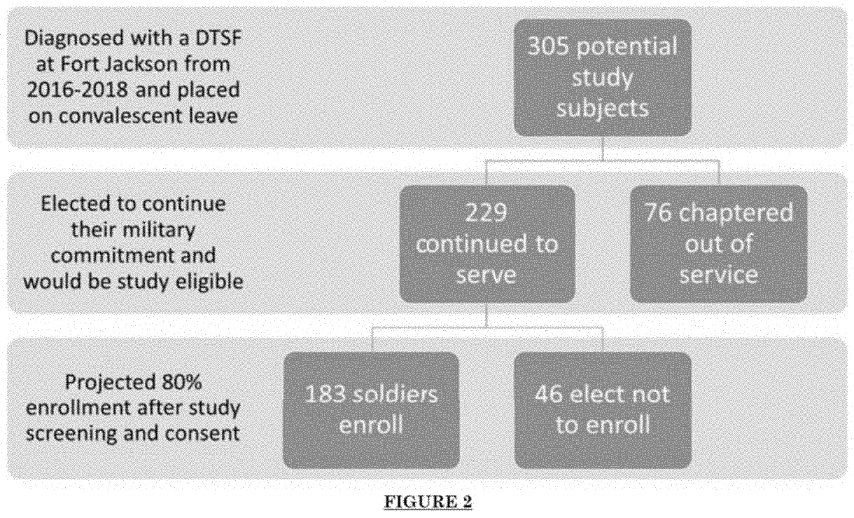

FIG. 3 shows participant outcome results.

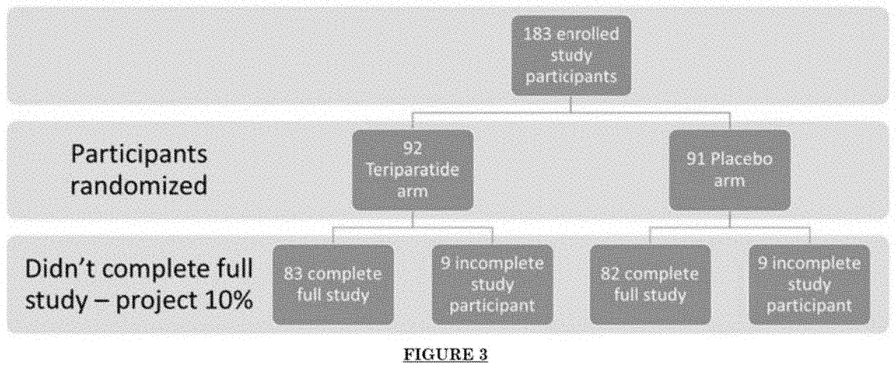

FIG. 4 shows a testing schedule for the current disclosure.

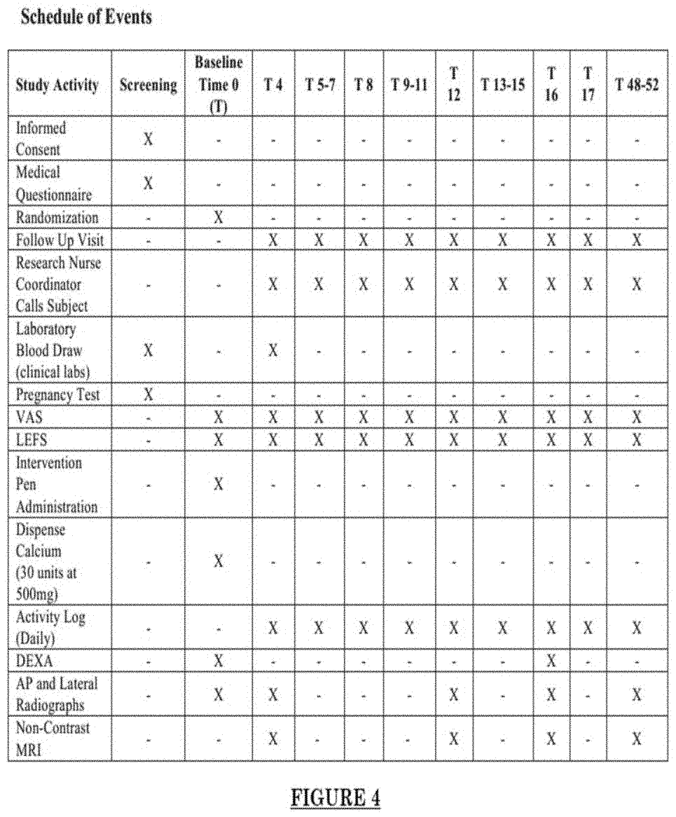

FIG. 5 shows Table 1 Summary of Hypotheses, Measurement Tools, and Data Analysis Procedures.

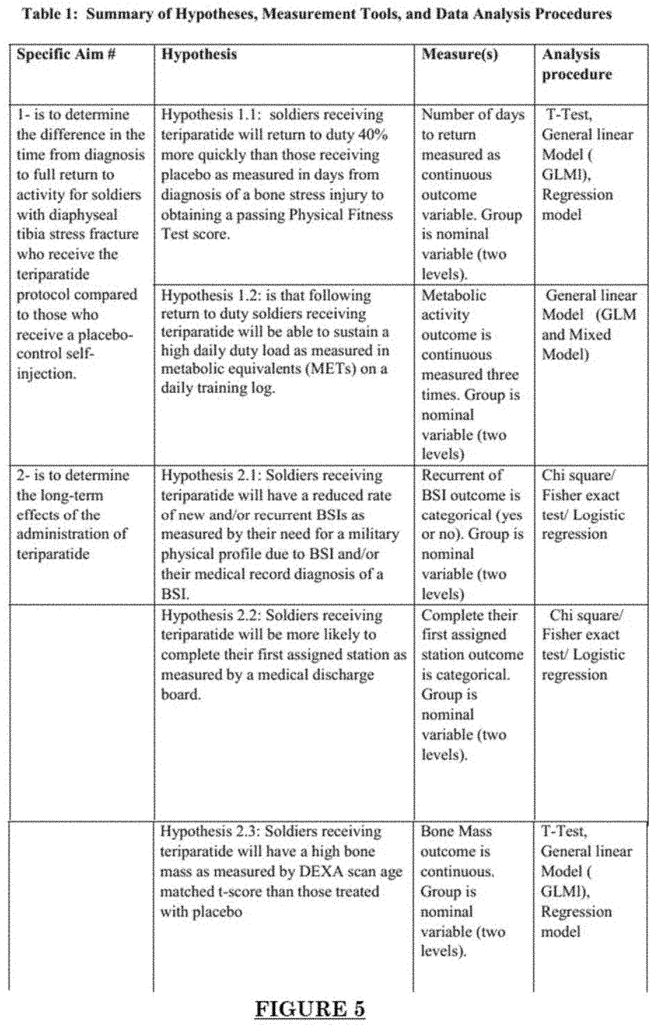

FIG. 6 shows Table 2 Required sample size for between group and within subjects with 80% power and alpha=0.05 for different effect sizes and RHO (intra class correlation).

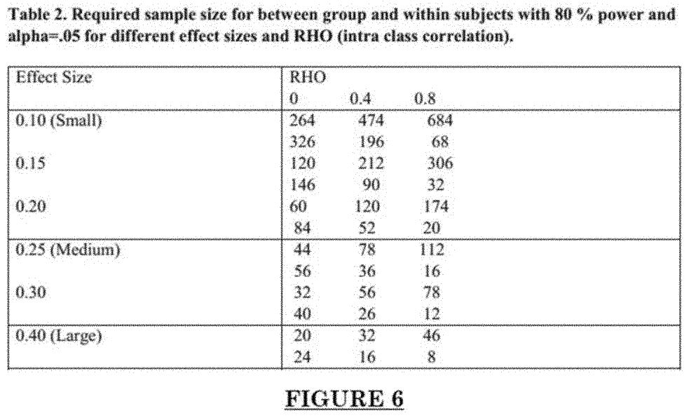

FIG. 7 shows Table 3 Required sample size for between group and within subjects interaction with 80% power and alpha=0.05 for different effect sizes.

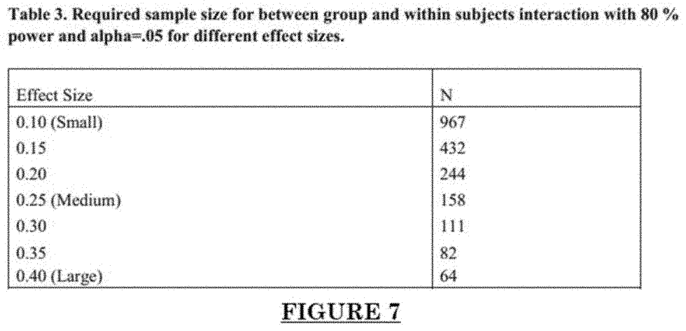

FIG. 8 shows results from usage of teriparatide therapy pursuant to the current disclosure in healing SBIs.

FIG. 9 shows plasma concentrations of teriparatide.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein inter-changeably and are meant to include the first and second indicated numbers and all the fractional and integral numerals there between.

The significance of the current disclosure is its evaluation of a novel, non-operative treatment approach for a prevalent and potentially career-ending injury affecting military trainees and elite, highly trained infantry soldiers. The economic cost of this injury to the military is profound, with some soldiers undergoing prolonged treatment requiring a medical separation board, during which their status must come under review for possible separation (discharge) from active military service. Soldiers who sustain a BSI in basic combat training are removed from training and placed in rehabilitation for an average of 62 days. In fact, the single most powerful medical predictor of discharge during Marine basic training is a BSI, with a four-fold higher rate of discharge in soldiers who suffer from a BSI, compared with those without this injury (Gun).

An attractive feature of the current disclosure is that it can be easily administered on-base or in-theater and will allow injured service members to remain with their units during recovery. Surgery or other invasive operative procedures are not required for this treatment, which also speeds recovery and recuperation time.

Teriparatide has been shown to be an anabolic bone agent that both increases bone production and bone mass. The administration of teriparatide (Forteo) assists the healing process by enabling the bodies' ability to rebalance the equation by directly promoting bone formation through osteoblast stimulation. Increased osteoblast activity improves bone mass quantity and quality. Teriparatide could significantly improve function improvement in patients following fracture.

Teriparatide, C¹⁸¹H₂₉₁N₅₅O₅₁S₂, H-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe-OH, a recombinant formulation of endogenous human parathyroid hormone (PTH) that has beneficial effects on the density, microarchitecture, and geometry of cancellous and cortical bones. This medication is currently indicated for patients who are at a high risk of fracture due to reduced bone density and includes: 1) postmenopausal women with osteoporosis, 2) men with primary or hypogonadal osteoporosis and, 3) both men and women with osteoporosis associated with sustained systemic glucocorticoid therapy (Forteo Product Label).

In previous studies on people in these populations teriparatide administration has led to clinically measurable increases in overall bone density, as well as increased rates of bone healing as measured by rate of enhancement of fracture callus, cortical thickening and bone mass (Cheng).

Longitudinal studies also suggest a persistent protective effect of teriparatide against new fractures while demonstrating an acceptable safety profile. One randomized, double blind, placebo-controlled study has tested the hypothesis that teriparatide accelerates fracture repair in post-menopausal women. The authors of the study concluded that fracture healing could be accelerated by teriparatide at the 20 mcg dose (Aspenberg). Members of our research team have used these findings to administer teriparatide at the 20 mcg dose to young adult NCAA collegiate athletes who have DTSFs. Our promising preliminary clinical observations in more than 100 NCAA athletes have led us to hypothesize that teriparatide will have a positive effect on bone healing and return to duty among service members with DTSFs.

There are theoretical risks with this medication. In rats, Forteo® (teriparatide [rDNA origin] injection) caused a dose-dependent increase in the incidence of osteosarcoma, a malignant bone tumor. Because of the uncertain relevance of the rat osteosarcoma finding to humans, the manufacturer recommends prescribing teriparatide only for patients for whom potential benefits outweigh potential risk (Forteo Package Insert, 2012). However, there have not been any reported cases of osteosarcoma in humans with over 16,000 unique patients that have been studied in a post-market analysis (Andrews).

Patients who could be at increased risk for malignant bone tumor will be excluded from the study. Based on the manufacturer's recommendations we will not prescribe teriparatide for patients at increased baseline risk for osteosarcoma. Increased risks include (Forteo Package Insert, 2012): Paget's disease of bone; Unexplained elevations of alkaline phosphatase (elevations in alkaline phosphatase may signal undiagnosed Paget's disease of bone)*; Pediatric and young adult patients with open epiphyses; Prior external beam or implant radiation therapy involving the skeleton. *Note: teriparatide treatment has been shown to elevate bone specific alkaline phosphatase as part of its mechanism of action; this elevation is expected.

Mechanism of Action

Human parathyroid hormone, an 84 amino acids peptide, is involved in the regulation of calcium and phosphate metabolism in bone and kidney. Low serum calcium levels stimulate the production of PTH, which acts by increasing the release of calcium from the bone and increasing renal calcium reabsorption. PTH also increases the intestinal absorption of calcium by formation of 1,25-dihydroxyvitamin D (Cheng). Therefore, chronic elevation of PTH, as in hyperparathyroidism, leads to an increase of bone resorption by the osteoclasts. In contrast, intermittent exposure to PTH, as daily injections of teriparatide, stimulates new bone formation by osteoblasts. This effect is called the “paradoxical effect of PTH”.

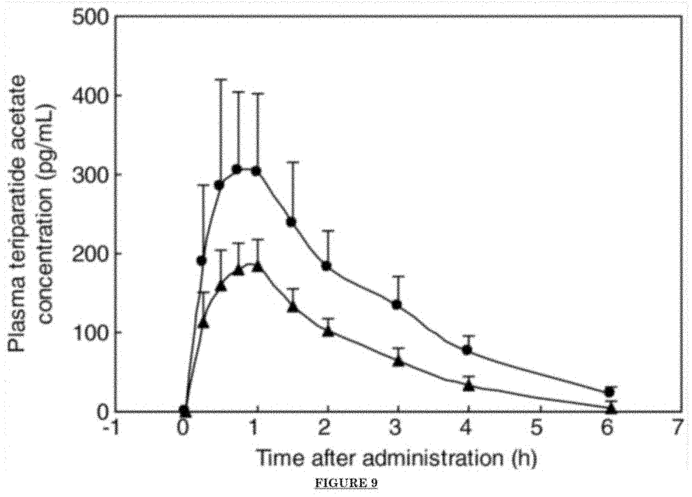

Intermittent exposure may be defined as a single treatment, which may be a single subcutaneous, oral, topical, mucosal, etc., treatment, as known to those of skill in the art, within a 24 hour period or a treatment regimen separated by a specified time interval, such as 2, 4, 6, 8, 10, 12, 14, 16, 18, 20, 22 hours, etc., including variations within these figures such as 1.5, 2.5, 3.5 hours, etc. Wherein the PTH level is allowed to “ebb and flow” such that PTH levels are not chronically elevated over a continual time-period but instead allow the half-life of PTH to remove it from the subject until the next treatment in order to trigger new bone formation via the paradoxical effect of PTH. As used herein “peak concentration (C_{MAX}) of PTH peptide in a blood plasma”, “area under concentration vs. time curve (AUC) of PTH peptide in a blood plasma”, “time to maximal plasma concentration (t_{MAX}) of PTH peptide in a blood plasma” are pharmacokinetic parameters known to one skilled in the art. Laursen, et al., Eur. J. Endocrinology 135:309-315, 1996. The “concentration vs. time curve” measures the concentration of PTH peptide in a blood serum of a subject vs. time after administration of a dosage of PTH peptide to the subject either by intranasal, intramuscular, subcutaneous, or other parenteral route of administration. “C_{MAX}” is the maximum concentration of PTH peptide in the blood serum of a subject following a single dosage of PTH peptide to the subject. “T_{MAX}” is the time to reach maximum concentration of PTH peptide in a blood serum of a subject following administration of a single dosage of PTH peptide to the subject. The current disclosure has discovered that one injection per twenty-four hours worked best. In one embodiment, the plasma concentration range may range from 100 picograms/ml up to 3000 picograms/ml. Following a subcutaneous injection, absorption of teriparatide was rapid with peak concentrations after 30 minutes and half-life was approximately 1 hour. Absolute bioavailability of teriparatide was 95%. Pharmacokinetic results were consistent for studies carried out in 1995-1997. In the remaining 8 studies, performed after 1997, the teriparatide concentrations were significantly lower. This could be due to the new batch of antibodies for the assay kit or to the change of the analytical site. FIG. 9 shows plasma concentrations of teriparatide. Mean changes of teriparatide acetate (in picograms per liter) in plasma after a single subcutaneous injection of teriparatide (filled circle 56.5 μg, filled triangle 28.2 μg) to 360 min. Bars represent standard deviation. The time course and the change in plasma teriparatide acetate concentrations are shown in FIG. 9. The plasma concentration of teriparatide increased in a dose-dependent manner, and Cmax was achieved 1 h after the injection (193.12±35.30 and 338.14±134.18 pg/mL and 28.2 and 56.5 μg groups, respectively). The remaining PK parameter data were AUCₗₐₛₜ 25.84±3.18 and 49.91±11.33 ng/min/mL, AUC_{inf} 28.07±2.47 and 52.73±10.03 ng/min/mL, Tmax 54.0±10.5 and 52.5±10.6 min, and T_{1/2} 69.57±13.04 and 77.69±35.22 min, in the 28.2 and 56.5 μg groups, respectively.

Teriparatide is a recombinant formulation of endogenous PTH, containing a 34 amino acid sequence which is identical to the N-terminal portion of this hormone. The pharmacologic activity of teriparatide is similar to the physiologic activity of PTH. The full-length PTH 1-84 peptide contains both the N-terminal and C-terminal regions of the hormone. The N-terminal region binds to PTH receptor 1 which is believed to confer all biological actions of PTH. The Cterminal region binds to another receptor (CPTHR), which responds exclusively to the C-terminal of PTH. It has been shown that the C-terminal fragments may have discrete biological properties acting on the osteocyte to enhance its apoptosis, accounting for the rationale for creating the N-terminal 1-34 PTH fraction (Cheng).

After subcutaneous injection, PTH analogues are absorbed rapidly. The bioavailability of PTH 1-34 and PTH 1-84 is 95 and 55%, respectively. The half-life after subcutaneous injection for PTH 1-34 is approximately 1 hour and that for PTH 1-84 is approximately 2.5 hours. The duration of action of PTH 1-84 is longer. For the present disclosure, PTH injections are given subcutaneously to avoid ingestion and breakdown of the hormone, thus oral, mucosal, or other types of treatment that would lead to breakdown of the PTH are not as effective as subcutaneous injections as used herein. Nor would PTH be administered via an IV (intravenously). An IV would lead to too rapid absorption of the PTH, which would result in the plasma concentration being too high and would also prevent the concentration from remaining at an effective therapeutic range long enough.

Included within the definition of biologically active peptides and proteins for use within the invention are natural or synthetic, therapeutically or prophylactically active, peptides (comprised of two or more covalently linked amino acids), proteins, peptide or protein fragments, peptide or protein analogs, and chemically modified derivatives or salts of active peptides or proteins. A wide variety of useful analogs and mimetics of PTH peptide are contemplated for use within the invention and can be produced and tested for biological activity according to known methods. Often, the peptides or proteins of PTH peptide or other biologically active peptides or proteins for use within the invention are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine. By aligning a peptide or protein analog optimally with a corresponding native peptide or protein, and by using appropriate assays, e.g., adhesion protein or receptor binding assays, to determine a selected biological activity, one can readily identify operable peptide and protein analogs for use within the methods and compositions of the invention. Operable peptide and protein analogs are typically specifically immunoreactive with antibodies raised to the corresponding native peptide or protein.

Teriparatide as an Osteoanabolic

Teriparatide leads to a rapid increase in bone formation markers, followed sometime thereafter by increases in bone resorption markers. PTH is likely to first stimulate bone formation, and only later promotes the processes associated with bone remodeling in which bone resorption predominates, and hence the concept of the “anabolic window” which is a period of time when the actions of PTH are maximally anabolic (Cheng).

The beneficial effects of teriparatide on bone density, microarchitecture, and geometry are seen in both cancellous and cortical bones. In cortical bones, it increases porosity in the inner one-third of bone, where the mechanical effect is minimal, and also improves bone microarchitecture and geometry at these sites. PTH stimulates periosteal apposition, which leads to an increase in cortical area, cortical thickness, and causes an overall increase in cross-sectional area.

Several preclinical studies have been conducted to evaluate the potential of teriparatide on fracture healing. They showed that once-daily administration of teriparatide enhanced callus formation and mechanical strength of fracture. Andreassen et al. were the first to study the healing of fractures in intact rats with tibial fractures after once-daily administration of PTH (1-34). Two doses of teriparatide were used; 60 and 200 kg/day. The efficacy was evaluated after 20 and 40 days of healing. The results showed an increased callus quantity and mechanical strength of fractures in rats treated with teriparatide, with a dose dependent response.

The only randomized, double-blind, placebo-controlled study to test the hypothesis that teriparatide accelerated fracture repair in humans, was reported in 2010. 102 postmenopausal women with distal radial fracture were randomized in a ratio of 1:1:1 as teriparatide 20 mcg, 40 mcg or placebo. Treatment was administered daily for eight (8) weeks. The primary efficacy variable was defined as the time to radiographic healing was 9.1, 7.4, and 8.8 weeks for placebo and teriparatide 20 mcg and 40 mcg, respectively. The results showed that there was not a statistically significant improvement in healing time at the dose of 40 mcg; however, the time to healing was shorter in the teriparatide 20 mcg group than in the placebo group. The lack of effect of the high dose of teriparatide (40 mcg) compared with placebo was incongruous with the results of preclinical studies in rats, in which higher doses were more potent for the healing of fractures.

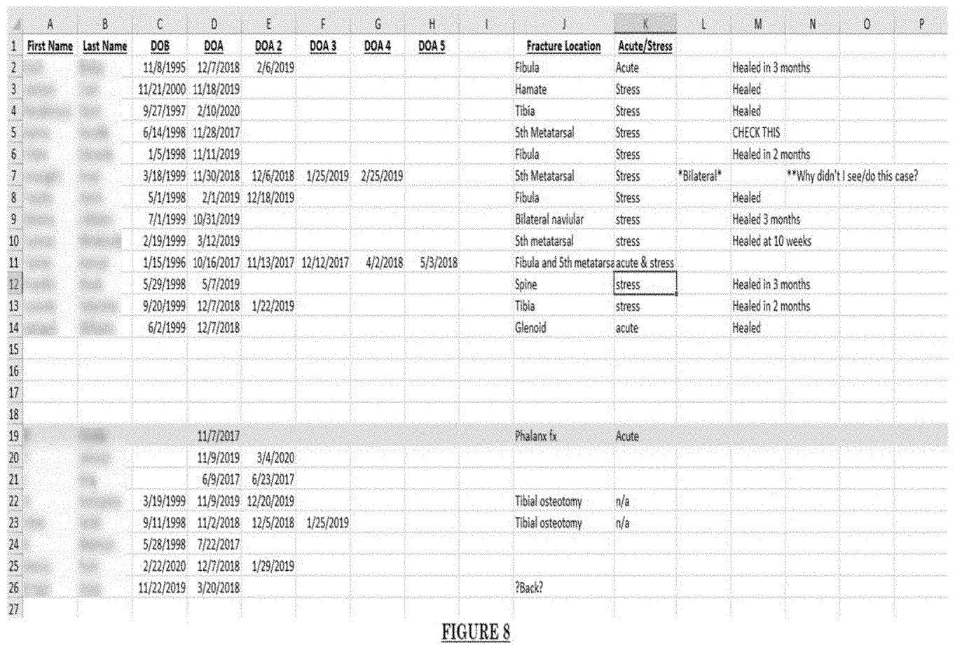

The current disclosure has administered teriparatide at the 20 mcg dose to young adult NCAA collegiate athletes who have DTSFs. Our promising clinical findings, see, e.g., FIG. 8, have led us to hypothesize that teriparatide will have a positive effect on bone healing and return to duty among service members with DTSFs. While a dosage of 20 mcg has been found effective, other dosages are considered within the scope of this application such as ranges from 5-50 mcg, 10-40 mcg, 15-35 mcg, 20-30 mcg, etc. In one embodiment, the preferred range is 10 mcg-40 mcg.

In 2009 it was reported the incidence of stress fractures among male infantry soldiers was 6.9% over the course of one year (Reynolds). Among the most common, yet difficult to treat BSIs, are those involving the diaphysis (shaft) of the tibia. Diaphyseal tibial stress fractures (DTSFs) require a long recovery time due to the anatomic and physiologic environment of the tibial diaphysis and the rigors placed on an active duty solider. Due to a lack of single timely and efficacious treatment, DTSFs are currently managed with numerous modalities. The mainstay of treatment is prolonged rest that typically requires, on average, seven months of limited or no duty. As a result, there have been many aimed interventions to attempt to accelerate bone growth in this area including pulsed ultrasound, pneumatic bracing, pulsed electromagnetic fields and diet modification. Unfortunately, and despite this wide array of treatment options, DTSFs continue to have a propensity for non-union and high potential for recurrence. This is a common condition that reduces duty service member readiness, performance and quality of life while increasing treatment costs and clinical workload for medical providers. Considering the enormous impact of DTSFs, the key problem to solve remains: finding new pathways that will accelerate the rate of bone healing and reducing recurrence rates in service members with DTSFs.

A specific feasibility analysis was performed at Fort Jackson, the enrollment site for this study. From 2016-2018 there were 305 persons diagnosed with a DTSF that were removed from training and placed on convalescent leave to accommodate healing. Other DTSFs that were able to manage a lower-grade injury and successfully graduate on time were filtered out of the 305. After the reduction of those that chaptered out of service, 229 recruits remained. Two additional trainees were recalled by their state, leaving a total of 227 injuries that would have been eligible as potential study candidates. Our fully powered study target is 176. 227 DTSFs would allow us to successfully complete our study enrollment over 3 years. The final year of the grant would be geared for the soldiers to complete follow-up DEXA scans.

We will study the troops who sustain the most severe form of DTSF that requires convalescent leave. These soldiers return to their home for 4 weeks of leave in order to heal. Often, these soldiers will be required to re-cycle through basic training, delaying their readiness and increasing their cost of training. Our primary outcome will be the soldier's ability and time until they pass their Physical Fitness Test (PFT). Evaluating a soldier's return to duty, as measured by the ability to successfully complete their PFT, is a practical and relevant marker of success after BSI. The PFT is uniformly acceptable as a marker of military physical readiness and easily translated across stations, deployment theaters and military service branches.

Our secondary outcome measure is the recurrence of BSI. This will be measured by: diagnosis of new or recurrent BSI, placement on military profile due to BSI, ability of the solider to complete their first station. Other clinical studies note the sustained improvement of bone mass and lower rate of recurrent fracture. A current USERM study is evaluating risk factors for BSI at Fort Jackson and will complete its enrollment and data collection within six months of the submission of this proposal and prior to the initiation of our study, if funded. The knowledge gained from this study by Dr. Beattie, a Co-Investigator on this study, will allow us to take the next steps into management of these injuries.

Given the novel ability of teriparatide to stimulate bone production through a non-operative, readily deployable and available medication this study may provide critical link to expedite troop readiness after sustaining a BSI. These common injuries delay, deter and/or force a medical discharge on too many of our soldiers. This leads to a potential lack of military readiness, increased human resource utilization for diagnosis, treatment and convalescence. The financial burden from BSIs is a staggering 100 million dollars per year to the military. Our comprehensive study team combining orthopedics, endocrinology and physical therapy allows us to care for the entire soldier, from BSI diagnosis through convalescence and recovery to a full return to duty.

Our comprehensive study team with access to the most vulnerable population for BSI in the world make our study feasible and have broad relevance to other BSIs in the both the military and civilian population.

Objectives/Specific Aims/Hypotheses

Objective: The project goals are to improve combat readiness of U.S. Soldiers and sustain the availability of the military to deploy by, 1) decreasing the number of days not physically ready for duty after diagnosis of a diaphyseal tibial stress fracture, 2) decreasing the need for a physical profile or medical discharge board after bone stress injury, and 3) decreasing the recurrence rates of bone stress injuries.

Specific Aim One is to determine the difference in the time from diagnosis to full return to activity for soldiers with diaphyseal tibia stress fracture who receive the teriparatide protocol compared to those who receive a placebo-control self-injection. We will address this aim by performing a prospective, longitudinal randomized, placebo-controlled trial in which 170 soldiers with acutely diagnosed diaphyseal tibia stress fractures will randomly receive a 1-month course of daily, self-injected 20 mcg. of teriparatide or daily, self-injected placebo in addition to current standard care.

Hypothesis 1.1: soldiers receiving teriparatide will return to duty 40% more quickly than those receiving placebo as measured in days from diagnosis of a bone stress injury to obtaining a passing Physical Fitness Test score.

Hypothesis 1.2 is that following return to duty soldiers receiving teriparatide will be able to sustain a high daily duty load as measured in metabolic equivalents (METS) on a daily training log.

Specific Aim Two is to determine the long-term effects of the administration of teriparatide. We will follow the study enrollees for 1-year to determine the incidence of drug related side effects, injury recurrence and/or additional bone stress injury based on data from the participants' Employee Medical Record.

Hypothesis 2.1: soldiers receiving teriparatide will have a reduced rate of new and/or recurrent bone stress injuries as measured by their need for a military physical profile due to bone stress injuries and/or their medical record diagnosis of a bone stress injury.

Hypothesis 2.2: soldiers receiving teriparatide will be more likely to complete their first assigned station as measured by a medical discharge board.

Hypothesis 2.3: Soldiers receiving teriparatide will have a high bone mass as measured by DEXA scan age matched t-score than those treated with placebo.

Successful completion of our aims will provide compelling evidence supporting teriparatide as a safe, non-operative and easily administered treatment option to accelerate recovery from DTSFs. The use of this drug will enhance the combat-readiness of America's soldiers.

Study Design

The study will examine the utility of a novel, non-operative, anabolic bone agent in the treatment of DTSFs. Teriparatide has been well studied in basic science and clinical studies to conclude its positive effects on building bone mass and preventing fractures in post-menopausal women.

This allowed the medication to gain it an FDA indication for treatment in these groups. We seek to explore additional uses for this anabolic agent in an at risk study population that has an imbalance of bone stress and bone formation resulting in a BSI. The only prior study to examine the effect of teriparatide on fracture healing demonstrated an absolute difference in the time to heal a fracture in the treatment groups, but was under powered to detect a difference. Given these positive results members of our research team have used these findings to administer teriparatide at the 20 mcg dose to young adult NCAA collegiate athletes who have DTSFs and other BSIs. Our promising preliminary clinical observations have led us to hypothesize that teriparatide will have a positive effect on bone healing and return to duty among service members with DTSFs.

A powered, prospective, controlled randomized study design will be utilized. The Fort Jackson Institutional Review Board (IRB) will oversee all compliance measures upon approval of the study. Any serious adverse effects, complications, or concerns will be reported immediately to the Fort Jackson IRB by the project staff. For transparency purposes, The University of South Carolina School of Medicine IRB will also be notified, but will rely on the Fort Jackson IRB to be the IRB of record.

Study subjects are to be recruited from the basic training corps at Fort Jackson via convenience sample. Soldiers who experience pain in the mid-shaft of the tibia during basic training will be evaluated and diagnosed according to the standard of care. Based on direct discussions with physical therapists stationed at Fort Jackson this diagnosis is often made by using a combination of bone scan and plain radiographs. After diagnosis, soldiers with a DTSF present to the physical therapy department for treatment and guidance for their progression of activities.

Once soldiers have been identified as having a DTSF and are being placed on convalescent leave they will be approached for enrollment in the study by the research nurse coordinator who will work full time at Fort Jackson. We have confirmed, based on information from 2016-2018 at Fort Jackson, that there is a sufficient number of DSTF that require convalescent leave that we should meet or exceed our enrollment target before the completion of the third year of the study.

Potential study subjects will have a thorough discussion of the risks and benefits to study participation and enrollment with a CITI certified research team member who is qualified to answer study related questions. A detailed discussion about the need to self-administer the study medication and the black box warning label for the risk of osteosarcoma for teriparatide will be conducted with the potential study enrollee. Additionally, we will discuss that teriparatide is being utilized for an indication that is not FDA approved. Finally, we will ask the soldier with a DTSF if they are planning to continue with the military commitment.

Soldiers who consent to the above will be considered potential study participants. Screening labs for study participation will include: total serum calcium, total serum alkaline phosphatase, 25-hydroxyvitamin D, Parathyroid Hormone, Creatinine clearance, Serum urine pregnancy test (females). Additionally, they will complete a medical history interview to ensure that they do not have any of the potential reasons for study exclusion in their medical history. After screening they will be considered study participants.

Inclusion Criteria

Soldiers actively enlisted in the U.S. Army attached to basic training unit at Fort Jackson;

Soldiers diagnosed with a tibial diaphyseal BSI requiring convalescent leave;

Skeletally mature;

Willing to self-administer study medication;

Desire to continue their military commitment;

Exclusion Criteria

History of any form of cancer;

Currently pregnant;

Paget's disease of bone;

Unexplained elevations of alkaline phosphatase (elevations in alkaline phosphatase may signal undiagnosed Paget's disease of bone);

Pediatric and young adult patients with open epiphyses;

Prior external beam or implant radiation therapy involving the skeleton;

Recent (within the last 6 months) urolithiasis (kidney stones);

Elevated serum calcium, alkaline phosphatase or uric acid;

Orthostatic hypotension;

Study participants will then be randomized from the study coordinating site at PHUSC Orthopedic center via computer block randomization. There will be a 1:1 randomization into either the placebo arm or the 20 mcg teriparatide treatment arm. Soldiers will then receive training on how to self-administer study medication in a supervised setting. Both arms will receive 500 mcg of supplemental calcium to take orally, daily. They will then be released for the four week convalescent leave.

After returning from convalescent leave the study participant will have their blood calcium level measured to ensure normal levels of serum calcium. The soldier will follow the standard practice at Fort Jackson by re-attaching to a unit and resume/re-start basic training. In addition, the soldier will report to the physical therapy department daily to monitor their progress with training. During their daily reporting they will complete a training log of their previous day's activities. This will allow for a day-to-day tracking of physiologic load. They will continue to advance the training and activity as tolerated based on their pain and guidance from the physical therapist (standard of care). The participant will also complete a Lower Extremity Functional Score (LEFs) at the time of study enrollment and then after they return from convalescent leave weekly until they successfully complete their Physical Fitness Test. Other standard orthopedic patient questionnaires/scales to be completed on a regular basis by the participant include the Short Form Survey (SF-36) and the Visual Analog Scale (VAS).

Participants will be followed with AP and Lateral radiographs of the tibia at standardized time points to assess boney healing. Additionally, MR images of the tibia will be obtained to assess the boney edema and healing compared with the index MRI at diagnosis.

Outcomes

Study outcomes have been chosen to focus the project on military relevant measures; a shortening of time to Return to Duty status, and the ability to sustain bone health. The Mission of the US Army is to, “Fight and win our Nation's wars by providing prompt, sustained land dominance” (Fort Jackson website). Our primary study performance measure is the soldier's ability to obtain a passing score on their Physical Fitness Test. This test is widely accepted as a surrogate for combat physical readiness. The ability to reduce the number of days from diagnosis of a BSI in a military trainee to combat readiness, as measured by a passing PFT score, is both critical and relevant to the military's ability to provide ‘prompt’ support to our Nation. Without the ability to successfully progress trainee to stations and/or deployments there can be unexpected drawdowns in troop availability.

The second outcome is the ability of the soldier to be able to provide ‘sustained’ fighting ability for our Nation. We will measure the sustaining effects of the study medication by monitoring the soldier's ability to perform at the first assigned station post treatment. Signs of an inability to perform full duties include: a physical profile, diagnosis of a new or recurrent BSI or in severe cases convening of a military discharge board. Each of these military specific and relevant outcomes would allow us to assess the impact of the study medication on the soldier's sustained ability to fight and win a war.

Safety Oversight

In order to maintain the highest levels of safety for this study and its participants a clinical safety oversight committee will be convened. Committee members will be knowledgeable in each area of the study including rehabilitation, bone healing and medication related side effects, respectively. This composition will allow expert knowledge of any possibly study related adverse events that could occur. Given the potential risks of the study medication and the expected accrual rate, the safety oversight committee would be scheduled to meet every six (6) months to review all adverse events, enrollment targets and any other study concerns that had been raised. In addition, they will meet on an ad hoc basis to discuss any serious adverse events (SAEs) that may occur. They oversight committee would be provided with a yearly stipend for their participation in the study.

This clinical safety oversight committee will be empowered to act on the best interest of the patient and for safety of the study subjects. If necessary, they would be empowered to close or suspend study enrollment due to SAE concerns or efficacy of the study. This would include if the study drug arm of the trail was significantly outperforming the placebo arm to stop the trail due to a lack of equipoise.

Reports from the clinical safety oversight committee would be provided to each PI at the conclusion of their meeting every 6 months. Action would then be taken by the study team to address any/all concerns.

In addition, an independent clinical trial monitoring organization will be contracted to ensure that all International Council on Harmonisation 6 and Good Clinical Practices compliance policies and practices are followed. The PIs, study team and clinical safety oversight committee will work with the clinical trial monitoring organization to develop a clinical monitoring plan that will include a communication plan, outline of visits types, monitoring activities and how corrective actions will be handled. The monitoring plan will include a site initiation visit, quarterly monitoring visits, potential for-cause visits, and a close out visit. The monitoring plan will also include details outlining how event reporting and/or action plans will be communicated to the IRB of record.

Statistical Plan and Data Analyses

The prospective, randomized, placebo controlled design of our study will significantly strengthen its scientific validity. We have performed an a priori power analysis with a projected effect size of a 40% reduction in days from diagnosis to a pass PFT. Results will be analyzed with an intention to treat analysis with respect to the primary and secondary outcomes.

The design for this study is a prospective with one of the outcomes (metabolic activity) measured three times per subject {baseline, 4 weeks, and 10 weeks} randomly assigned to two groups: placebo (standard treatment) and intervention (receiving teriparatide). In addition to the study subjects, the data analyst in this study will be blinded to the group assignment. The analysis discussion is based on the assumption of equal sample sizes of the groups.

Analysis for Specific Aim 1 and 2:

Descriptive statistics will be computed on the variables. For categorical variables, the univariate constructions will include frequency distributions. For continuous variables statistics will be included measure of central tendency (mean and median) and measure of spread (standard deviation and range). Our outcomes in this study are continuous (number of days from diagnosis, metabolic activity, and bone mass) and categorical (recurrent BSI, first assigned station, and bone cancer). The descriptive statistics for outcomes will be done by group. Bivariate analysis includes Chi-square, Fisher exact test, Student's t-test, and correlations will be used to examine relationship between our outcomes and demographic and selected variables. Multivariate analysis including multiple regression, General Linear Model (GLM), and logistic regression will use to examine the relationships between sets of the predictors on outcomes variable(s). In the analysis for outcome measured over time, expected mean squares will be calculated and the appropriate combination will be used for hypothesis tests with specific functions of the repeated measures. General linear model analyses in SAS (Statistical Analysis System) (GLM and MIXED procedures) will be used to exam the effects of: 1) Time, 2) Treatment, and 3) Time by treatment interaction. Since this is a repeated measures design, the intraclass correlation (RHO) is assumed to be positive and constant across all repeated measures. One advantage of using Mixed model is that it uses incomplete data in the data analyses. In addition, parameter estimates of the effects of covariates and of the appropriate structure for the repeated observations will be estimated. Several techniques will be used to check the four major assumptions (linearity, independence, homoscedasticity, and normality) in the regression models.

Data will be analyzed using intent to treat methods. Missing values for outcomes will be examined both the extent and pattern of missingness. Should missing value up to 10%; we will use multiple imputation method (MI) to replace the missing value (Cohen & Cohen 2003). We will use sensitivity analyses for dropouts to determine if the dropouts are systematic, completely at random, or missing at random. If a dropout is not dependent on both observed and unobserved variables it would be completely at random. If a dropout is related to unobserved variable it would be considered missing at random. Several techniques will be used to examine the pattern of dropouts (90). For example, a dummy variable will be created for dropout and non-dropout and correlated with other variables in the data set. A significant strong relationship would indicate data are not missing at random, and our statistician will assess and adjust for the extent of nonrandomness. The level of statistical significance will be set at 0.05. Values that will be reported include the adjusted R-square value, beta coefficients, standard error of beta, and Pvalue. Level of statistical significance: 0.05 (see Table 1).

Sample Size and Power Calculation

For continuous variables measured one time: Effect size (f) is defined as ½ delta. Effect sizes values are typically in the range of 0-1. In social science applications, values of delta=0.1, 0.25, and 0.4 or greater correspond to “small”, “medium”, and “large” effects (Cohen 2003). For multiple regression (hypothesis 2-4), effect size (f2) will be defined as R2/1+R2. Where R2 is the amount of variance is explained for outcome by the sets of predictors. Effect size (f2)=0.02, 0.15, and 0.35 or greater correspond to “small”, “medium”, and “large” effects (Cohen 1988). For USC-676-P (1426) chi square test, the effect size W=0.1, 0.3, and 0.5 or greater correspond to “small”, “medium”, and “large” effects (Cohen).

For continuous variable measure several times: we are examining three effects: 1) Between group (group effect), 2) Within subject (Time effect), and 3) Between-Within interaction effect (Time by Group interaction). Therefore, the power analysis is done for each effect (table 2 and 3). Since this is a repeated measures design, the intraclass correlation (RHO) is assumed to be positive and constant across all repeated measures. The power calculation indicates that there is at least 80% power for alpha=0.05, medium effect size (f=0.25), Rho ranges from 0 to 0.5, and for n=172 for between-subject effects. Also, the power calculation indicate that there are at least 90% power for alpha=0.05, between small and medium effect size (f=0.15), Rho ranges from 0 to 0.8, and for n=190 for within-subject effect. In addition the power calculation indicate that there are at least 80% power for alpha=0.05, medium effect size (f=0.25), and for n=158 for interaction of between-subjects and within-subjects group. The power calculation for multiple regression shows that we will have at least 80% power for alpha=0.01 and having ten predictors variables in the model with total sample size of 835 for small effect size, 131 for medium effect size, and 69 for large effect size. A sample size of 180 achieves at least 80% power to detect medium effect size using 3 degrees of freedom Chi-Square Test with a significance level (alpha) of 0.01 (tables 2 and 3).

SPSS (25.0) and SAS (9.4) will be used to set up, enter and analyze the data. All data will be entered and will be kept in the safe place. To check the accuracy of data entry, a random sample of entered data (50%) will be printed and verified with the hard copy. If there is data entry error (more than 5%) all questionnaires will be reentered and rechecked for accuracy. Several backups will be made.

Data Management

Investigators will assist with completion of all data collection and will be on site to describe the proper implementation of the study. No one who is not connected with the study will have access to the data. All study personnel will have current certification regarding the protection of human subjects and will sign a confidentiality statement.

Access to Target Population/Enrollment Strategy

Our study team has met with the Deputy Commander of the Moncrief Medical Center on Ft. Jackson who has expressed his support of this project. In addition, he has supported Major Bower PT, PhD, and Major Stoltenberg PT, PhD, active duty physicals therapists who serve as leaders in the Physical Therapy Department of Moncrief Medical Center, to serve as Co-Investigators on the study. We believe that the support of officers in the command and control structure at Ft. Jackson combined with active duty officers who currently treat patients with DTSFs at Ft. Jackson will allow our study team to have an excellent working relationship with the soldiers at Ft. Jackson who sustain the injury targeted in our study.

We have begun the military IRB process to ensure approval of the proposed clinical trial Teriparatide: STRONG. We understand that with this vulnerable population that there are many stakeholders who seek to ensure the safe execution of the proposed clinical trial. We have secured support from the Deputy Commander of Moncrief Medical Center. We have initiated the approval from the Institutional Review Board at Fort Gordon and begun to secure approval of the personnel at U.S. Army Training and Doctrine Command (TRADOC).

We intend to have an embedded full time grant funded research nurse coordinator positioned in the physical therapy department of Moncrief Medical Center, the location of treatment of soldiers with DTSFs, to oversee the enrollment and administrative duties. This on base study team member will ensure a direct access point to soldiers, base leadership, therapists, potential study subjects and enrolled study subjects to members of the study team. This will provide an immediate ability of the study team to address any concerns and ensure that no potential study subjects are missed.

Major Stoltenberg, PT, Chief Physical Therapy Services at Fort Jackson reported that from 2016-2018 they have seen 305 tibial stress fractures that were severe enough the soldier was placed on convalescent leave. After filtering out soldiers who chaptered out of service there were 229. This number would represent our target population and represents under 3 years' worth of data.

Given this real time, at our study enrollment site historical information, we believe that we will be well within our study target number and enrollment rate. These numbers reflect cases where tibial stress injury was the primary diagnosis. Analyzing tibial stress injury as a secondary diagnosis resulted in eight more cases annually (after accounting for those that chaptered out).

Our strong working relationship with Ft. Jackson, close proximity, active duty physical therapists currently stationed at Ft. Jackson on our study team provide a solid foundation of access to our target population. Additionally, our knowledge of the current rates of DTSFs that require convalescent leave at Ft. Jackson are greater than our target study enrollment based on our power analysis can assuage concerns about an inability to complete study enrollment.

Should a national crisis occur, all study enrollment will be suspended until the threat has passed. If a local emergency takes place, and access to Fort Jackson changes, it will be suggested to the Department of the Army that the trial be moved to another base in a neighboring state, such as Fort Bragg or Fort Benning, to maintain consistency of the target study population.

Upon completion of this successful project, where it will be determined if treatment with teriparatide enables a 40% reduction in the standard treatment time for DTSFs, the Primary Investigator will disseminate the results in academic journals, military medical knowledge share outlets, meetings, and/or conferences.

FIG. 8 shows experimental results of treatment with teriparatide as per the description above. As FIG. 8 shows, treatment with teriparatide resulted in healing of the subjects' stress fractures. The PTH peptide can also be administered in conjunction with other therapeutic agents such as bisphonates, calcium, vitamin D, estrogen or estrogen-receptor binding compounds, selective estrogen receptor modulators (SERMs), bone morphogenic proteins and/or calcitonin. In one embodiment, 500 mg calcium may be administered daily for the duration of treatment with teriparatide. Vitamin D may also be administered along with teriparatide. Unless a patient is hypercalemic, defined as a total serum calcium concentration >10.4 mg/dL (>2.60 mmol/L) or ionized serum calcium >5.2 mg/dL (>1.30 mmol/L), calcium would be used as part of the treatment regimen. For patients with low vitamin D concentrations, defined as a 25(OH)D concentration of 12 to 20 ng/mL (30 to 50 nmol/L), Vitamin D may also be administered during treatment descried herein.

One dosage regimen found particularly effective with respect to the treatments shown in FIG. 8 was administering between 10 mcg-40 mcg, such as 20 mcg, for a duration of at least twenty-eight (28) days. In some circumstances, this treatment regimen can be continued for up to six (6) months for more severe/persistent injuries.

In a further embodiment, during treatment of a stress fracture, one may administer 20 mcg subcutaneously daily for 28 days then re-evaluate for pain and radiographic changes. If there is no significant improvement in one or both, the treatment regimen may continue for another 28 days and re-assess. Additionally calcium levels, vitamin d levels are checked along with alkaline phosphate and a basic metabolic panel. If alkaline phosphate levels are considered high, which may be indicative of Paget's Disease and/or bone cancer, the patient would be contraindicated for treatment as described herein. They may be afflicted with a bone turnover disease.

In one embodiment, teriparatide may be administered in a dose comprising a 0.025% w/v aqueous solution of teriparatide (20 μg/80 μl dose) in acetate buffer pH 4, preserved with 0.3% w/v metacresol. It may be presented in a 3 ml cartridge assembled into a disposable product-dedicated pen injector that contains treatment for 28 days. The pen-injector is identical to that approved for Humalog-Pen™ in June 1997 (EU/1/97/0042/001), with the exception of injection volume and dose display. The product does not contain ingredients of animal or human origin.

With respect to prior art efforts using teriparatide with respect to osteoporosis, the duration of the current treatment regimen is shorter and more targeted. The current disclosure administers teriparatide for 28 days then re-assesses the patient. The current disclosure also has a more specific target for treatment success, fracture healing, with defined x-ray and or ct parameters. The current disclosure treatment regimen would not be effective for osteoporosis because the treatment duration is not long enough to build bone throughout the body. The current disclosure takes advantage of the hyperemic effect of a fracture to supplement the body's desire to build more bone by providing an additional hormonal signal to ensure that enough bone is built and that it is done more quickly by harnessing the body's own healing mechanisms.

The present disclosure provides for non-operative, quicker recovery, much quicker pain relief, quicker return to full activity, quicker return to activities of daily living, quicker return to work/duty, lower cost, less invasive, more reliable healing, enhancing the body's natural response, and provides a natural treatment with a more robust healing response.

Amino Acid Sequences

Teriparatide Length 34—SVSEIQLMHNLGKHLNSMERVEWLRKKLQDVHNF

Sequence Listing Free Text

Teriparatide—Length 34—Ser Val Ser Glu Ile Gln Leu Met His Asn Leu Gly Lys His Leu Asn Ser Met Glu Arg Val Glu Trp Leu Arg Lys Lys Leu Gln Asp Val His Asn Phe

In yet additional aspects of the invention, a kit for treatment of a mammalian subject comprises a stable pharmaceutical composition of one or more PTH peptide compound(s) formulated for subcutaneous delivery to the mammalian subject wherein the composition is effective for treating or preventing bone stress injuries. The kit further comprises a pharmaceutical reagent vial to contain the one or more PTH peptide compounds. The pharmaceutical reagent vial is composed of pharmaceutical grade polymer, glass or other suitable material. The pharmaceutical reagent vial is, for example, a silanized glass vial. The kit further comprises a needle or other delivery device for delivery of the composition subcutaneously to a subject. The delivery device may be composed of a pharmaceutical grade polymer, glass or other suitable material.

All patents, patent applications, published applications, and publications, databases, websites and other published materials referred to throughout the entire disclosure herein, unless noted otherwise, are incorporated herein by reference in their entirety.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A treatment for bone stress injuries comprising:
administering a therapeutically effective amount of an endogenous human parathyroid hormone to stimulate new bone formation via osteoblasts;
wherein the treatment avoids operative treatment and is anabolic.

2. The treatment of claim 1, wherein the endogenous human parathyroid hormone comprises teriparatide.

3. The treatment of claim 1, wherein the endogenous human parathyroid hormone is administered in a dosage ranging from 10 mcg to 40 mcg.

4. The treatment of claim 1, wherein the endogenous human parathyroid hormone is administered intermittently.

5. The treatment of claim 4, wherein the endogenous human parathyroid hormone is administered at least once every twenty-four hours.

6. The treatment of claim 1, herein the endogenous human parathyroid hormone is administered subcutaneously.

7. The treatment of claim 1, wherein the endogenous human parathyroid hormone has an amino acid sequence as shown in SEQ ID NO:1.

8. The treatment of claim 1, wherein the treatment provides improvements in bone density, bone microarchitecture and bone geometry in both cancellous and cortical bones.

9. A method for treatment of bone stress injuries comprising:
administering a therapeutically effective amount of teriparatide to increase bone production and bone mass via osteoblasts;
wherein the method does not include operative treatment and is anabolic.

10. The method of claim 9, wherein the method promotes bone formation through osteoblast stimulation.

11. The treatment of claim 9, wherein the endogenous human parathyroid hormone is administered in a dosage ranging from 10 mcg to 40 mcg.

12. The treatment of claim 9, wherein teriparatide is administered intermittently.

13. The treatment of claim 12, wherein teriparatide is administered at least once every twenty-four hours.

14. The treatment of claim 9, wherein teriparatide is administered subcutaneously.

15. The treatment of claim 9, wherein teriparatide has an amino acid sequence as shown in SEQ ID NO:1.

16. The treatment of claim 9, wherein the treatment provides improvements in bone density, bone microarchitecture and bone geometry in both cancellous and cortical bones.

17. A prophylactic method for protecting against new and recurrent bone stress injuries comprising:
administering a therapeutically effective amount of an endogenous human parathyroid hormone;
wherein the treatment avoids operative treatment and is anabolic.

18. The treatment of claim 17, wherein the endogenous human parathyroid hormone is administered in a dosage ranging from 10 mcg to 40 mcg.

19. The treatment of claim 18, wherein teriparatide is administered subcutaneously and intermittently.

20. The treatment of claim 19, wherein teriparatide is administered at least once every twenty-four hours.


SEQUENCE LISTING (1 sequence)

SEQ ID NO: 1 - protein, 34 aa, Homo sapiens
SVSEIQLMHNLGKHLNSMERVEWLRKKLQDVHNF